(19) United States
(12) Patent Application Publication
(10) Pub. No.: US 2025/0143561 A1
(43) Pub. Date: May 8, 2025

(54) IMAGE PROJECTION DEVICE, VISION TEST DEVICE, AND FUNDUS PHOTOGRAPHY DEVICE

(71) Applicant: QD LASER, INC., Kawasaki-shi, Kanagawa (JP)

(72) Inventor: Makoto SUZUKI, Kawasaki-shi (JP)

(73) Assignee: QD LASER, INC., Kawasaki-shi, Kanagawa (JP)

(21) Appl. No.: 18/837,874

(22) PCT Filed: Feb. 6, 2023
(86) PCT No.: PCT/JP2023/003804

§ 371 Date: Aug. 12, 2024

(30) Foreign Application Priority Data
Mar. 28, 2022 (JP) 2022-051969

(51) Int. Cl. A61B 3/00; A61B 3/06; A61B 3/12

(52) U.S. Cl. CPC A61B 3/0008; A61B 3/063; A61B 3/1225

(57) ABSTRACT

An image projection device includes a light source, a scanning unit that scans a light beam emitted from the light source, and an optical system that converges a plurality of light beams, which are emitted from the scanning unit at different times, at a convergence point in an eye of a user and then irradiates a retina of the user with the plurality of light beams to project an image, wherein diameters of the plurality of light beams incident on a cornea of the user are adjusted to 0.36 mm or greater and 0.46 mm or less when a half angle of a larger one of a horizontal angle of view and a vertical angle of view of an area where the retina is irradiated with the plurality of light beams at the convergence point is 10° or greater and 30° or less.

### TECHNICAL FIELD

The present invention relates to an image projection device, a vision test device, and a fundus photography device.

### BACKGROUND ART

An image projection device using Maxwellian view is known in which a scanned light beam is converged in an eye and then applied onto a retina (for example, Patent Documents 1 and 2). Further, a vision test device using Maxwellian view is also known (for example, Patent Document 3).

### PRIOR ART DOCUMENT

### Patent Document

Patent Document 1: Japanese Patent Application Laid-Open No. 2018-116219
Patent Document 2: Japanese Patent Application Laid-Open No. 2014-102368
Patent Document 3: International Publication No. 2019/069578

### SUMMARY OF THE INVENTION

### Problem to be Solved by the Invention

In the Maxwellian view, a plurality of light beams emitted from the scanning unit at different times are converged at a convergence point in the eye of the user or the like and then applied onto the retina. In this case, it is desired that the half angle of the larger one of the horizontal angle of view and the vertical angle of view of the area where the retina is irradiated with the plurality of light beams at the convergence point in the eye be 10° or greater. Even when the retina is irradiated with a plurality of light beams at such an angle of view, it is desirable that the diameters of the plurality of light beams on the retina fall within a predetermined range at any position of the retina.

The present invention has been made in view of the above problem, and an object of the present invention is to make the diameters of a plurality of light beams on a retina fall within a predetermined range when the half angle of the larger one of the horizontal angle of view and the vertical angle of view of the area where the retina is irradiated with the plurality of light beams is 10° or greater.

### Means for Solving the Problem

The present invention is an image projection device including: a light source; a scanning unit that scans a light beam emitted from the light source; and an optical system that converges a plurality of light beams, which are emitted from the scanning unit at different times, at a convergence point in an eye of a user and then irradiates a retina of the user with the plurality of light beams to project an image, wherein diameters of the plurality of light beams incident on a cornea of the user are adjusted to 0.36 mm or greater and 0.46 mm or less when a half angle of a larger one of a horizontal angle of view and a vertical angle of view of an area where the retina is irradiated with the plurality of light beams at the convergence point is 10° or greater and 30° or less.

In the above configuration, a configuration in which the plurality of light beams have diameters of 55 μm or greater and 77 μm or less on the retina may be employed.

In the above configuration, a configuration in which numerical apertures of the plurality of light beams when entering the cornea of the user are approximately zero regardless of the user may be employed.

In the above configuration, a configuration in which the plurality of light beams incident on the cornea of the user have diameters of 0.38 mm or greater and 0.44 mm or less may be employed.

In the above configuration, a configuration in which the plurality of light beams are monochromatic light beams of red light beams, green light beams, or blue light beams, or combined light beams obtained by combining at least two of a red light beam, a green light beam, and a blue light beam may be employed.

The present invention is a vision test device including: a light source; a scanning unit that scans a light beam emitted from the light source; an optical system that converges a plurality of light beams, which are emitted from the scanning unit at different times, at a convergence point in an eye of a subject and then irradiates a retina of the subject with the plurality of light beams; and an input unit to which a response of the subject to the plurality of light beams applied to the retina is input, wherein diameters of the plurality of light beams are adjusted to 0.36 mm or greater and 0.46 mm or less when a half angle of a larger one of a horizontal angle of view and a vertical angle of view of an area where the retina is irradiated with the plurality of light beams at the convergence point is 10° or greater and 30° or less.

In the above configuration, a configuration in which the plurality of light beams have diameters of 55 μm or greater and 77 μm or less on the retina may be employed.

In the above configuration, a configuration in which the subject responds to each of the plurality of light beams sequentially applied to the retina by operating the input unit may be employed.

The present invention is a fundus photography device including: a light source; a scanning unit that scans a light beam emitted from the light source; an optical system that converges a plurality of light beams, which are emitted from the scanning unit at different times, at a convergence point in an eye of a subject and then irradiates a retina of the subject with the plurality of light beams; a detector that detects the plurality of light beams reflected by the retina; and an acquisition unit that acquires a fundus image of the subject from the plurality of light beams detected by the detector, wherein, when a half angle of a larger one of a horizontal angle of view and a vertical angle of view of an area where the retina is irradiated with the plurality of light beams at the convergence point is 10° or greater and 30° or less, diameters of the plurality of light beams incident on the cornea of the subject are adjusted to 0.36 mm or greater and 0.46 mm or less.

In the above configuration, a configuration in which the plurality of light beams have diameters of 55 μm or greater and 77 μm or less on the retina may be employed.

### Effects of the Invention

According to the present invention, when the half angle of the larger one of the horizontal angle of view and the vertical angle of view of the area where the retina is irradiated with a plurality of light beams is 10° or greater, the diameters of the plurality of light beams on the retina can be adjusted to fall within a predetermined range.

### BRIEF DESCRIPTION OF THE DRAWINGS

FIG. 1 is a block diagram of an image projection device in accordance with a first embodiment.

FIG. 2 illustrates an optical system of the image projection device in accordance with the first embodiment.

FIG. 3 illustrates a light beam in the first embodiment.

FIG. 4 is a diagram illustrating a method of generating an image in the first embodiment.

FIG. 5(a) to FIG. 5(d) are graphs presenting results of Simulation 1.

FIG. 6(a) to FIG. 6(c) are graphs presenting simulation results of the spot diameter of a light beam with respect to the angle α when a light beam of a single wavelength is used as the light beam.

FIG. 7(a) to FIG. 7(d) are graphs presenting results of Simulation 2.

FIG. 8 is a graph presenting simulation results of the spot diameter of a light beam with respect to a cornea incident diameter of the light beam.

FIG. 9(a) and FIG. 9(b) are graphs presenting results of Simulation 3.

FIG. 10 is a block diagram of a vision test device in accordance with a second embodiment.

FIG. 11 illustrates an optical system of the vision test device in accordance with the second embodiment.

FIG. 12 is a flowchart illustrating an example of a testing method of the vision test device in accordance with the second embodiment.

FIG. 13(a) to FIG. 13(c) are views for describing an image for testing projected onto the retina in the flowchart of FIG. 12.

FIG. 14 is a block diagram of a fundus photography device in accordance with a third embodiment.

FIG. 15 illustrates an optical system of the fundus photography device in accordance with the third embodiment.

FIG. 16 is a flowchart illustrating an example of a testing method of the fundus photography device in accordance with the third embodiment.

### MODES FOR CARRYING OUT THE INVENTION

Hereinafter, embodiments of the present invention will be described with reference to the drawings.

### FIRST EMBODIMENT

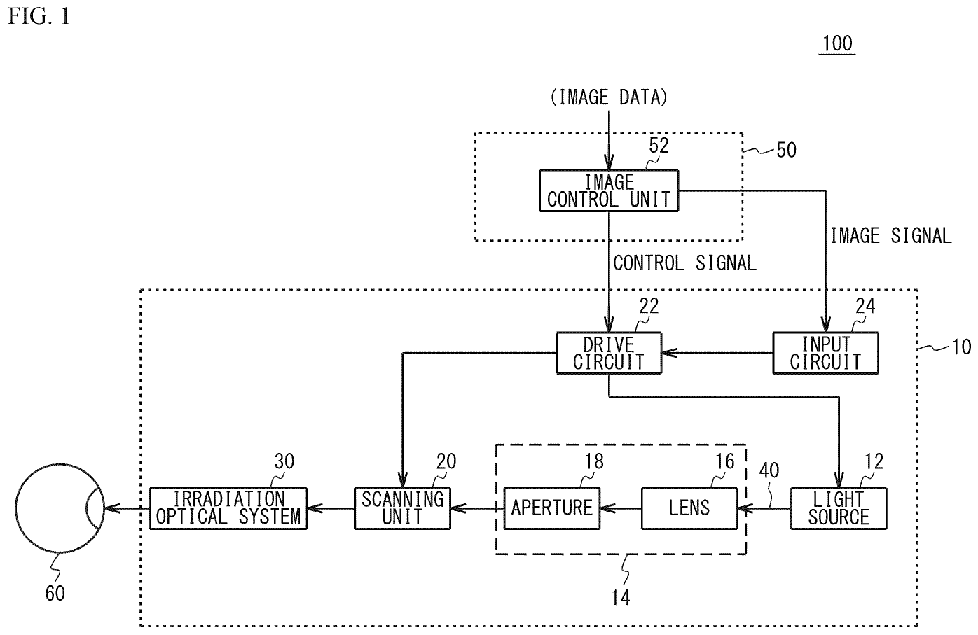

FIG. 1 is a block diagram of an image projection device 100 in accordance with a first embodiment. As illustrated in FIG. 1, the image projection device 100 includes a projection unit 10 and a control unit 50. The projection unit 10 includes a light source 12, an adjustment unit 14 including a lens 16 and an aperture 18, a scanning unit 20, a drive circuit 22, an input circuit 24, and an irradiation optical system 30. The control unit 50 includes an image control unit 52.

Image data is input to the image control unit 52 from a camera and/or a recording device (not illustrated). The image control unit 52 generates an image signal based on the input image data and outputs the image signal to the input circuit 24. The drive circuit 22 drives the light source 12 and the scanning unit 20 based on the control signal of the image control unit 52 and the image signal acquired by the input circuit 24.

The light source 12 emits a light beam 40 (laser beam) that is a visible light beam of, for example, a red laser beam (wavelength: about 610 nm to 660 nm), a green laser beam (wavelength: about 515 nm to 540 nm), and a blue laser beam (wavelength: about 440 nm to 480 nm). The light source 12 that emits red, green, and blue laser beams is, for example, a light source in which laser diode chips for red, green, and blue (RGB) and a three color combining device are integrated. The light source 12 may emit the light beam 40 of a single wavelength.

The adjustment unit 14 shapes the light beam 40. The scanning unit 20 (scanner) is, for example, a scanning mirror such as a micro electric mechanical system (MEMS) mirror or a transmissive scanner, and scans the light beam 40 in two-dimensional directions. The irradiation optical system 30 irradiates an eye 60 of the user with the scanned light beam 40.

The image control unit 52 may be configured so that a processor such as a central processing unit (CPU) performs processing in cooperation with a program. The image control unit 52 may be a specially designed circuit. The image control unit 52 may project an image input from a camera installed at an appropriate position toward the visual line direction of the user on the eye 60 of the user. The image control unit 52 may project an image input from a recording device or the like or superimpose a camera image and an image from a recording device or the like to project so-called augmented reality (AR).

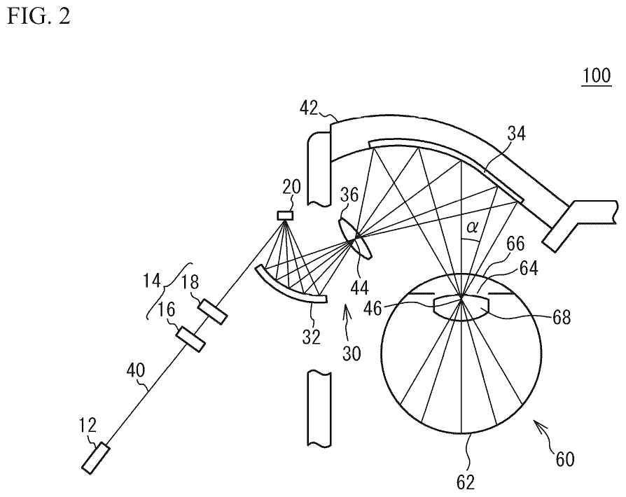

FIG. 2 illustrates an optical system of the image projection device 100 in accordance with the first embodiment. As illustrated in FIG. 2, the image projection device 100 is a retinal projection type head-mounted display using Maxwellian view in which the light beam 40 for causing the user to visually recognize an image is directly applied onto a retina 62 of the user.

The light source 12 emits the light beam 40 based on the control of the image control unit 52 (see FIG. 1). The light beam 40 emitted by the light source 12 passes through the lens 16. The lens 16 is a condenser lens that converts the light beam 40 from diffusion light to convergent light. The diameter of the light beam 40 transmitted through the lens 16 is adjusted by the aperture 18. The light beam 40 that has passed through the aperture 18 enters the scanning unit 20. The scanning unit 20 scans the light beam 40 in two-dimensional directions, i.e., the horizontal direction and the vertical direction.

A plurality of the light beams 40 that are scanned in the two-dimensional directions by the scanning unit 20 and are emitted from the scanning unit 20 in different directions at different times enter the irradiation optical system 30. The irradiation optical system 30 includes a reflection mirror 32, a projection mirror 34, and a lens 36. The components of the light source 12, the adjustment unit 14, the scanning unit 20, and the irradiation optical system 30 are fixed to, for example, a spectacle-type frame 42.

The light beams 40 emitted from the scanning unit 20 enter the reflection mirror 32. The reflection mirror 32 is a concave mirror having a reflection surface formed of a curved surface such as a free-form surface, and has a positive condensing power. The light beams 40 reflected by the reflection mirror 32 converge at a convergence point 44 in front of the projection mirror 34. The lens 36 is provided at the convergence point 44. The lens 36 is, for example, a biconvex lens. The light beams 40 pass through the lens 36 and enter the projection mirror 34.

The projection mirror 34 is disposed in front of the eye 60 of the user. The projection mirror 34 is a concave mirror having a reflection surface formed of a curved surface such as a free-form surface, and has a positive condensing power. The projection mirror 34 reflects the light beams 40 toward the eye 60 of the user. The light beams 40 reflected by the projection mirror 34 pass through a pupil 64 of the eye 60 of the user, converge at a convergence point 46 in the eye 60, and then are applied onto the retina 62. The convergence point 46 is located, for example, in or near a crystalline lens 68. The user can visually recognize an image by irradiating the retina 62 with the light beams 40.

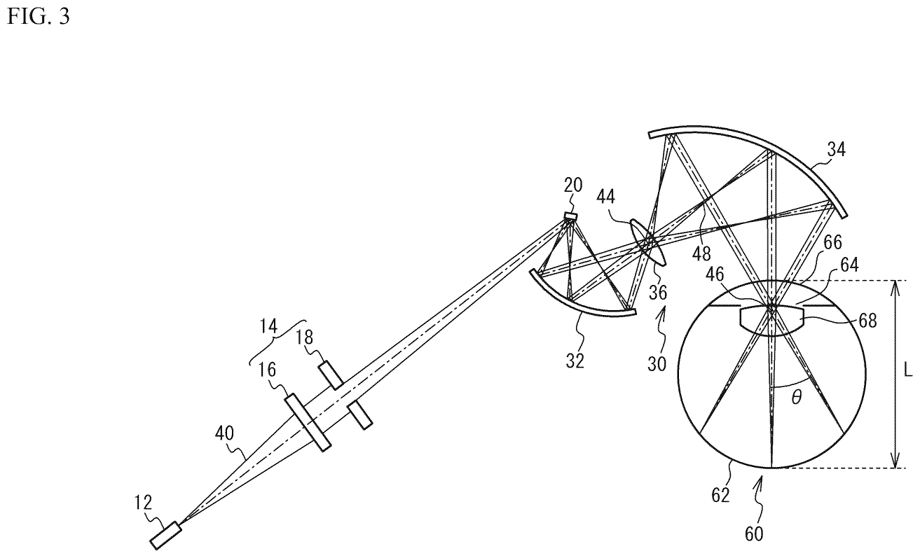

FIG. 3 is a diagram illustrating the light beam 40 in the first embodiment. As illustrated in FIG. 3, the light beam 40 emitted by the light source 12 passes through the lens 16. The lens 16 is a condenser lens that converts the light beam 40 from diffusion light to convergent light. The diameter of the light beam 40 transmitted through the lens 16 is adjusted by the aperture 18. The aperture 18 has an opening that blocks a part of the light beam 40 and allows the rest to pass through. The opening is fixed in size and has, for example, a substantially circular shape. The diameter of the opening is adjusted so that the diameter of the light beam 40 when the light beam 40 enters a cornea 66 of the user is within a range of 0.36 mm to 0.46 mm. That is, the diameter of the light beam 40 when the light beam 40 enters the cornea 66 of the user is within a range of +0.05 mm from a median of 0.41 mm in actual projection.

The light beam 40 that has passed through the aperture 18 enters the scanning unit 20 in a state of convergent light. A plurality of the light beams 40, which are scanned in the two-dimensional directions by the scanning unit 20 and are emitted from the scanning unit 20 in different directions at different times, enter the reflection mirror 32. Each of the light beams 40 is condensed before the reflection mirror 32, and then becomes diffusion light to enter the reflection mirror 32. Since the reflection mirror 32 has a positive condensing power, each of the light beams 40 is reflected by the reflection mirror 32, and thus is converted from diffusion light into substantially parallel light. The lens 16 is provided between the light source 12 and the scanning unit 20 so that the light beam 40 reflected by the reflection mirror 32 becomes substantially parallel light.

The light beams 40 reflected by the reflection mirror 32 converge at the convergence point 44 in front of the projection mirror 34. The lens 36 is provided at the convergence point 44. The lens 36 is a condensing lens that converts each of the light beams 40 from substantially parallel light to convergent light. Each of the light beams 40 transmitted through the lens 36 is condensed at a condensing point 48 in front of the projection mirror 34, and then becomes diffusion light and enters the projection mirror 34.

Since the projection mirror 34 has a positive condensing power, each of the light beams 40 is reflected by the projection mirror 34, thereby being converted from diffusion light into substantially parallel light, and enters the eye 60 of the user. Thus, the numerical aperture of each of the light beams 40 is approximately zero when the light beams 40 enter the cornea 66 of the eye 60. This does not change depending on the user wearing the image projection device 100. The diameters of the light beams 40 when entering the cornea 66 are 0.36 mm to 0.46 mm (0.41 mm+0.05 mm). The lens 36 is provided at the convergence point 44 so that the light beams 40 reflected by the projection mirror 34 become substantially parallel light.

The light beams 40 converge at the convergence point 46 within the eye 60 of the user. Each of the light beams 40 is converted from substantially parallel light to convergent light by the crystalline lens 68 and is focused in the vicinity of the retina 62. A half angle θ of the larger one of the horizontal angle of view and the vertical angle of view of the area where the retina 62 is irradiated with the light beams 40 at the convergence point 46 in the eye 60 is 10° or greater and 30° or less. In other words, the larger one of the angles in the horizontal direction and the vertical direction between the light beam 40 positioned at the center of the irradiation area where the retina 62 is irradiated with the light beams 40 (corresponding to the light beam 40 corresponding to the center of the projection image projected on the retina 62) and the light beam 40 positioned at the edge of the irradiation area (corresponding to the light beam 40 corresponding to the edge of the projection image) is 10° or greater and 30° or less. In the first embodiment, the area where the retina 62 is irradiated with the light beams 40 is longer in the horizontal direction than in the vertical direction (for example, image projection in which the ratio of the length in the vertical direction to the length in the horizontal direction is 9:16), and the half angle θ of the horizontal angle of view is 10° or greater and 30°.

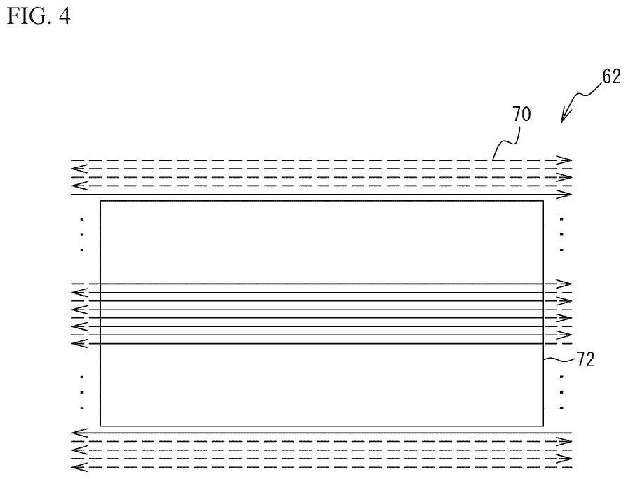

FIG. 4 is a diagram illustrating a method of generating an image in the first embodiment. As illustrated in FIG. 4, the scanning unit 20 raster-scans the light beam 40 on the retina 62 from the upper left to the lower right as indicated by arrows 70. This projects an image 72 onto the retina 62. The area where the retina 62 is irradiated with the light beams 40 is longer in the horizontal direction than in the vertical direction, for example, and the image 72 projected on the retina 62 is a horizontally long image with an aspect ratio of 9:16, for example. Even when the scanning unit 20 is driven, the light beam 40 is not applied onto the retina 62 unless the light source 12 emits the light beam 40. For example, the light beam 40 is not emitted at the dashed arrows 70 in FIG. 4. The drive circuit 22 synchronizes the emission of the light beam 40 from the light source 12 with the driving of the scanning unit 20. This causes the light source 12 to emit the light beam 40 in a predetermined area (the solid arrows 70) on the retina 62.

### [Simulation 1]

The diameter of the light beam 40 on the retina 62 was simulated, the light beam 40 being emitted to the retina 62 at an angle α (see FIG. 2) with respect to the light beam 40 located at the center of the irradiation area, in a case where the irradiation area where the retina 62 is irradiated with the light beams 40 is longer in the horizontal direction than in the vertical direction, and the half angle θ of the horizontal angle of view at the convergence point 46 is 30°. The simulation conditions are as follows. Hereinafter, the diameter of the light beam 40 when the light beam 40 enters the cornea 66 is referred to as a cornea incident diameter of the light beam 40, and the diameter of the light beam 40 on the retina 62 is referred to as a spot diameter of the light beam 40.

### Simulation Conditions:

Light beam 40: White light beam that is a combination of a red laser beam (wavelength: 640 nm), a green laser beam (wavelength: 520 nm), and a blue laser beam (wavelength: 465 nm)
Axial length (length L between the cornea 66 and the retina 62: see FIG. 3): 23 mm, 24 mm, 25 mm, 26 mm
Cornea incident diameter of the light beam 40: 0.25 mm, 0.5 mm, 1 mm, 2 mm, 4 mm

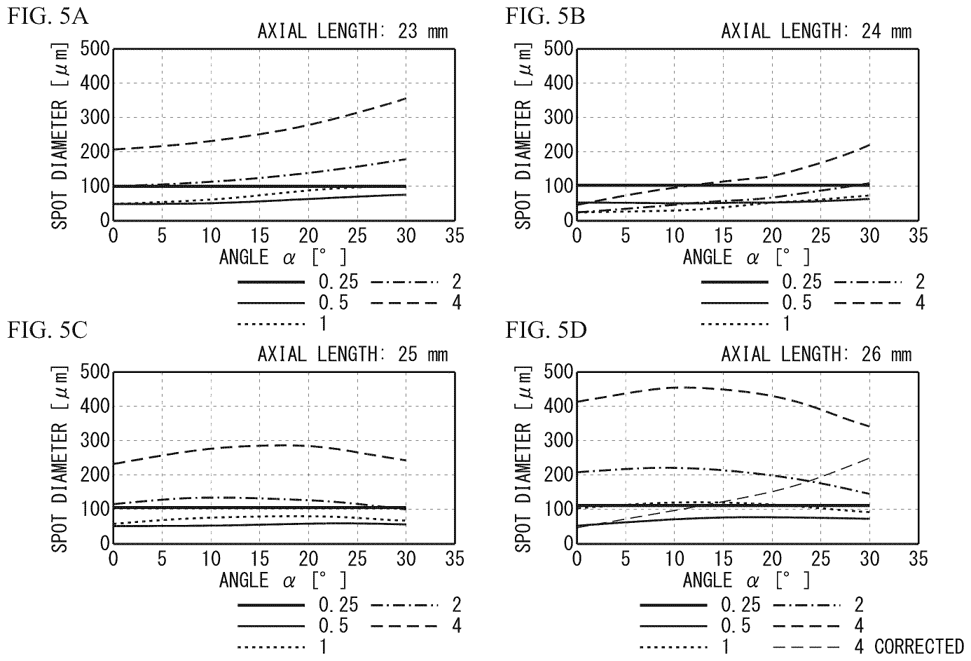

FIG. 5(a) to FIG. 5(d) are graphs presenting the results of Simulation 1. In FIG. 5(a) to FIG. 5(d), the horizontal axis represents the angle α [°], and the vertical axis represents the spot diameter [μm] of the light beam 40. The cornea incident diameters of the light beam 40 of 0.25 mm, 0.5 mm, 1 mm, 2 mm, and 4 mm are respectively indicated by a thick solid line, a solid line, a dotted line, a dash-dotted line, and a broken line. FIG. 5(a) presents the results when the axial length is 23 mm, FIG. 5(b) presents the results when the axial length is 24 mm, FIG. 5(c) presents the results when the axial length is 25 mm, and FIG. 5(d) presents the results when the axial length is 26 mm. In FIG. 5(d), the thin broken line indicates the case where the correction is performed so that the spot diameter of the light beam 40 is minimized when the angle α is 0° in the case where the cornea incident diameter of the light beam 40 is 4 mm.

As presented in FIG. 5(a) to FIG. 5(d), it is understood that, when the axial length is 23 mm, the cornea incident diameter of the light beam 40 is large, i.e., 2 mm and 4 mm, and the angle α is 10° or greater, the spot diameter of the light beam 40 greatly changes. On the other hand, it is found that, by adjusting the cornea incident diameter of the light beam 40 to 0.25 mm to 1 mm, the variation in the spot diameter of the light beam 40 is kept small in the range of the angle α of 0° to 30° in any case where the axial length is 23 mm, 24 mm, 25 mm, or 26 mm. It is also understood that even when the axial length changes to 23 mm, 24 mm, 25 mm, and 26 mm, the variation in the spot diameter of the light beam 40 when the angle α is 0° can be kept small by adjusting the cornea incident diameter of the light beam 40 to 0.25 mm to 1 mm. From these facts, it can be said that the Near Field state in which the light beam 40 is thin and the Far Field state in which the light beam 40 is thick are mixed in FIG. 5(a) to FIG. 5(d).

Therefore, the results of Simulation 1 reveal that, by adjusting the cornea incident diameter of the light beam 40 to 0.25 mm to 1 mm, even when the axial length is different, the spot diameter itself of the light beam 40 can be kept small while keeping the variation thereof small in the range of the angle α of 0° to 30°.

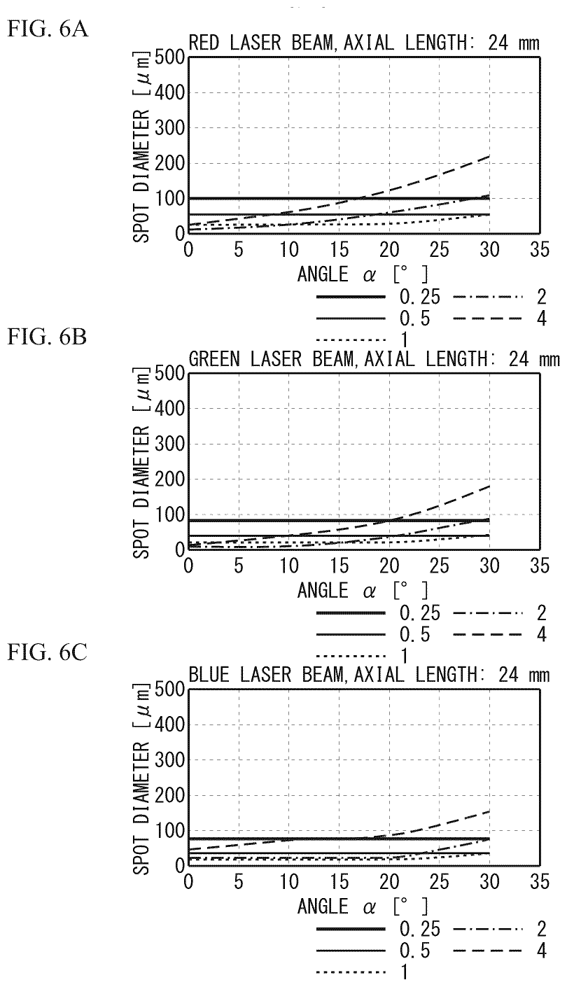

Although FIG. 5(a) to FIG. 5(d) illustrate the case where a white light beam obtained by combining a red laser beam, a green laser beam, and a blue laser beam is used as the light beam 40, the case where a light beam of a single wavelength of a red laser beam, a green laser beam, or a blue laser beam is used as the light beam 40 will be described below. FIG. 6(a) to FIG. 6(c) presents simulation results of the spot diameter of the light beam 40 with respect to the angle α when a light beam of a single wavelength is used as the light beam 40. In FIG. 6(a) to FIG. 6(c), the horizontal axis represents the angle α [°], and the vertical axis represents the spot diameter [μm] of the light beam 40. The cornea incident diameters of the light beam 40 of 0.25 mm, 0.5 mm, 1 mm, 2 mm, and 4 mm are indicated by a thick solid line, a solid line, a dotted line, a dash-dotted line, and a broken line, respectively. FIG. 6(a) presents the results when the axial length is 24 mm and a red laser light with a wavelength of 640 nm is used as the light beam 40, FIG. 6(b) presents the results when the axial length is 24 mm and a green laser beam with a wavelength of 520 nm is used as the light beam 40, and FIG. 6(c) presents the results when the axial length is 24 mm and a blue laser beam with a wavelength of 465 nm is used as the light beam 40.

It is understood that the variation in the spot diameter of the light beam 40 with respect to the angle α has the same tendency between the case where the light beam 40 is a light beam of a single wavelength of a red laser beam, a green laser beam, or a blue laser beam in FIG. 6(a) to FIG. 6(c) and the case where the light beam 40 is a white light beam obtained by combining a red laser beam, a green laser beam, and a blue laser beam as presented in FIG. 5(b). It is understood that the spot diameter of the light beam 40 when the white light beam obtained by combining the red laser beam, the green laser beam, and the blue laser beam is used as the light beam 40 (FIG. 5(b)) corresponds to the largest size of the spot diameter of the light beam 40 when the light beam of a single wavelength of the red laser beam, the green laser beam, or the blue laser beam is used as the light beam 40 (FIG. 6(a) to FIG. 6(c)).

### [Simulation 2]

The results of Simulation 1 reveal that the cornea incident diameter of the light beam 40 is preferably 0.25 mm to 1 mm. Therefore, the spot diameter of the light beam 40 with respect to the angle α was simulated when the cornea incident diameter of the light beam 40 was finely changed in a range of 0.25 mm to 1 mm. The simulation conditions are as follows.

### Simulation Conditions:

Light beam 40: White light beam obtained by combining a red laser beam (wavelength: 640 nm), a green laser beam (wavelength: 520 nm), and a blue laser beam (wavelength: 465 nm)
Axial length: 23 mm, 24 mm, 25 mm, 26 mm
Cornea incident diameter of the light beam 40: 0.3 mm, 0.4 mm, 0.5 mm, 0.6 mm, 0.7 mm

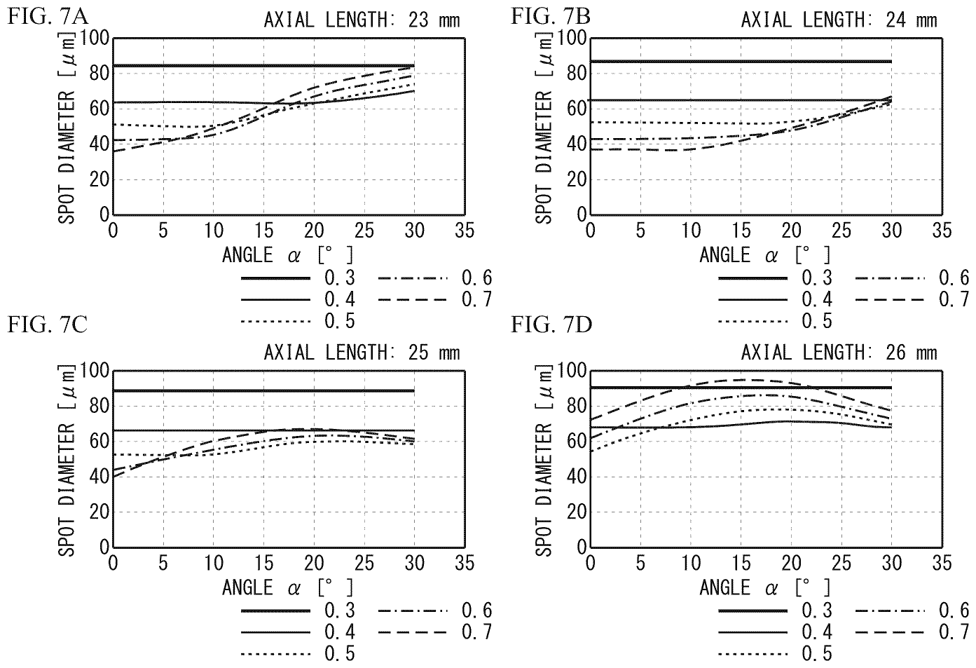

FIG. 7(a) to FIG. 7(d) are graphs presenting the results of Simulation 2. In FIG. 7(a) to FIG. 7(d), the horizontal axis represents the angle α [°], and the vertical axis represents the spot diameter [μm] of the light beam 40. The cornea incident diameters of the light beam 40 of 0.3 mm, 0.4 mm, 0.5 mm, 0.6 mm, and 0.7 mm are indicated by a thick solid line, a solid line, a dotted line, a dash-dotted line, and a broken line, respectively. FIG. 7(a) presents the results when the axial length is 23 mm, FIG. 7(b) presents the results when the axial length is 24 mm, FIG. 7(c) presents the results when the axial length is 25 mm, and FIG. 7(d) presents the results when the axial length is 26 mm.

As presented in FIG. 7(a) to FIG. 7(d), in the case that the cornea incident diameter of the light beam 40 is 0.3 mm, the variation in the spot diameter of the light beam 40 is small even when the axial length and the angle α are changed, but the spot diameter of the light beam 40 itself is increased. In the case that the cornea incident diameter of the light beam 40 is 0.7 mm, the variation in the spot diameter of the light beam 40 due to the changes in the axial length and the angle α is large, and in the case that the axial length is 26 mm, the spot diameter of the light beam 40 is larger than in the case that the cornea incident diameter of the light beam 40 is 0.3 mm.

In the case that the axial length is 23 mm (FIG. 7(a)), when the angle α is 0° to 30°, the spot diameter of the light beam 40 is 42 μm to 79 μm by adjusting the cornea incident diameter of the light beam 40 to 0.4 mm to 0.6 mm, and the spot diameter of the light beam 40 is 50 μm to 74 μm by adjusting the cornea incident diameter to 0.4 mm to 0.5 mm. In the case that the axial length is 24 mm (FIG. 7(b)), when the angle α is 0° to 30°, the spot diameter of the light beam 40 is 42 μm to 65 μm by adjusting the cornea incident diameter of the light beam 40 to 0.4 mm to 0.6 mm, and the spot diameter of the light beam 40 is 52 μm to 65 μm by adjusting the cornea incident diameter to 0.4 mm to 0.5 mm.

In the case that the axial length is 25 mm (FIG. 7(c)), when the angle α is 0° to 30°, the spot diameter of the light beam 40 is 44 μm to 66 μm by adjusting the cornea incident diameter of the light beam 40 to 0.4 mm to 0.6 mm, and the spot diameter of the light beam 40 is 52 μm to 66 μm by adjusting the cornea incident diameter to 0.4 mm to 0.5 mm. In the case that the axial length is 26 mm (FIG. 7(d)), when the angle α is 0° to 30°, the spot diameter of the light beam 40 is 55 μm to 85 μm by adjusting the cornea incident diameter of the light beam 40 to 0.4 mm to 0.6 mm, and the spot diameter of the light beam 40 is 55 μm to 78 μm by adjusting the cornea incident diameter to 0.4 mm to 0.5 mm.

Therefore, it is found that, by adjusting the cornea incident diameter of the light beam 40 to 0.4 mm to 0.6 mm, the spot diameter of the light beam 40 is 42 μm to 85 μm in the case that the axial length is 23 mm to 26 mm and the angle α is 0° to 30°. It is also found that, by adjusting the cornea incident diameter of the light beam 40 to 0.4 mm to 0.5 mm, the spot diameter of the light beam 40 is 42 μm to 78 μm in the case that the axial length is 23 mm to 26 mm and the angle α is 0° to 30°.

Here, simulation results of the spot diameter of the light beam 40 with respect to the cornea incident diameter of the light beam 40 are presented. The simulation conditions are as follows.

### Simulation Conditions:

Light beam 40: White light beam obtained by combining a red laser beam (wavelength: 640 nm), a green laser beam (wavelength: 520 nm) and a blue laser beam (wavelength: 465 nm)
Axial length: 24 mm

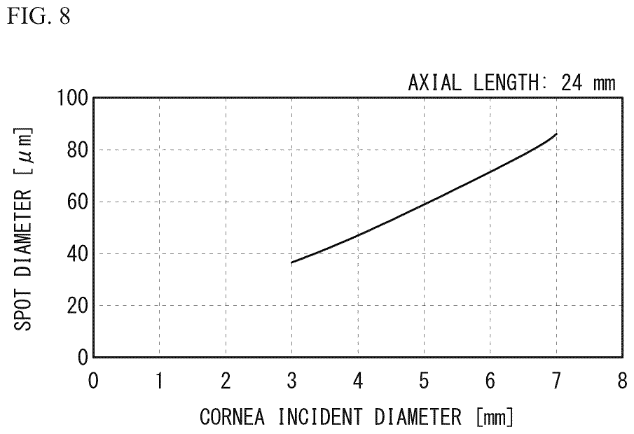

FIG. 8 is a graph presenting a simulation result of the spot diameter of the light beam 40 with respect to the cornea incident diameter of the light beam 40. In FIG. 8, the horizontal axis represents the cornea incident diameter [mm] of the light beam 40, and the vertical axis represents the spot diameter [μm] of the light beam 40. FIG. 8 presents the spot diameter of the light beam 40 in the case where the cornea incident diameter of the light beam 40 is 3 mm to 7 mm. The case where the cornea incident diameter of the light beam 40 is 3 mm to 7 mm corresponds to the case where light passes through the pupil 64 and is applied onto the retina 62 in natural vision, and particularly, the case where the cornea incident diameter is about 7 mm corresponds to the case where light passes through the pupil 64 and is applied onto the retina 62 in a dark place. As the cornea incident diameter of the light beam 40 increases, the chromatic aberration on the retina 62 increases, and thus the Far Field state is more dominant with respect to the spot diameter of the light beam 40. The spot diameter of the light beam 40 when the cornea incident diameter of the light beam 40 is 7 mm is approximately 85 μm. The spot diameter of the light beam 40 also has approximately the same value when a broadband light source is used.

Therefore, in consideration of the diameter of the light beam 40 when the light beam 40 is applied onto the retina 62 in natural vision, the spot diameter of the light beam 40 is preferably kept 85 μm or less regardless of the axial length and the position on the retina 62. From the result of Simulation 2, the spot diameter of the light beam 40 can be adjusted to 42 μm to 85 μm by adjusting the cornea incident diameter of the light beam 40 to 0.4 mm to 0.6 mm. Further, by adjusting the cornea incident diameter of the light beam 40 to 0.4 mm to 0.5 mm, the spot diameter of the light beam 40 can be adjusted to 42 μm to 78 μm.

A large variation in the spot diameter of the light beam 40 with respect to the change in the angle α is not preferable for the user to obtain a uniform resolution feeling with respect to the projection image. The amount of change in the energy density of the light beam 40 on the retina 62 is preferably kept 3 dB or less, and the amount of variation in the spot diameter of the light beam 40 is preferably kept 1.5 dB or less.

From the results of Simulation 2, it is understood that the cornea incident diameter of the light beam 40 is preferably around 0.4 mm in order to keep the spot diameter of the light beam 40 85 μm or less and to keep the variation in the spot diameter of the light beam 40 with respect to the changes in the axial length and the angle α small.

### [Simulation 3]

As described in Simulation 2 above, the cornea incident diameter of the light beam 40 is preferably around 0.4 mm. In the case that the cornea incident diameter of the light beam 40 is 0.4 mm, the spot diameter of the light beam 40 is about 65 μm when the axial length is 24 mm and the angle α is 0°. When the spot diameter of the light beam 40 is used as a reference, the spot diameter of the light beam 40 is required to be within a range of 55 μm to 77 μm in order that the amount of variation in the spot diameter of the light beam 40 is 1.5 dB or less, in other words, ±0.75 dB or less. Therefore, the spot diameter of the light beam 40 with respect to the angle α was simulated when the cornea incident diameter of the light beam 40 was finely changed around 0.4 mm. The simulation conditions are as follows.

### Simulation Conditions:

Light beam 40: White light beam obtained by combining a red laser beam (wavelength: 640 nm), a green laser beam (wavelength: 520 nm), and a blue laser beam (wavelength: 465 nm)
Axial length: 23 mm, 26 mm
Cornea incident diameter of the light beam 40: 0.34 mm, 0.36 mm, 0.38 mm, 0.4 mm, 0.42 mm, 0.44 mm, 0.46 mm

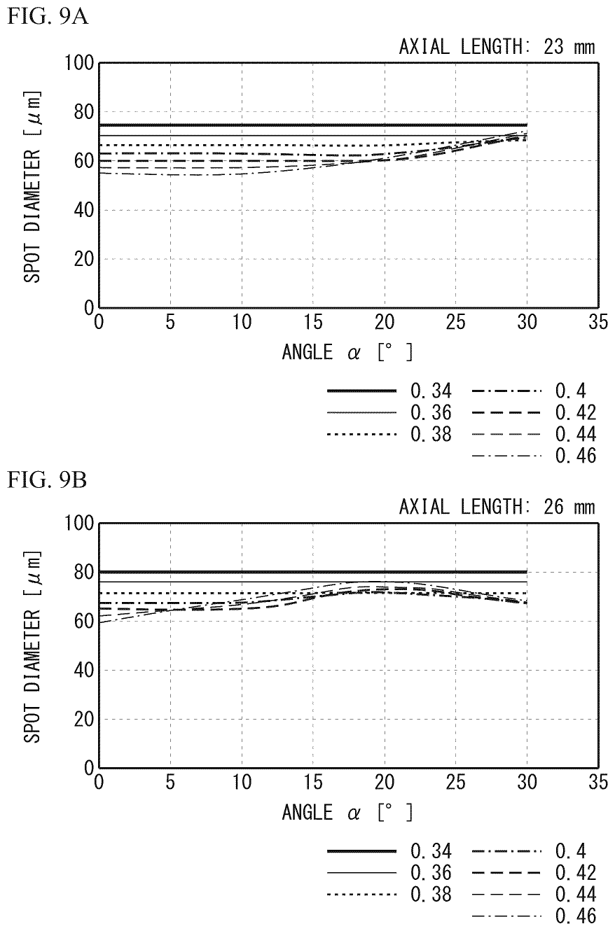

FIG. 9(a) and FIG. 9(b) are graphs presenting results of Simulation 3. Tables 1 and 2 are tables presenting the results of Simulation 3. In FIG. 9(a) and FIG. 9(b), the horizontal axis represents the angle α [°], and the vertical axis represents the spot diameter [μm] of the light beam 40. The cornea incident diameters of the light beams 40 of 0.34 mm, 0.36 mm, 0.38 mm, 0.4 mm, 0.42 mm, 0.44 mm, and 0.46 mm are indicated by a thick solid line, a solid line, a dotted line, a thick dash-dotted line, a thick broken line, a broken line, and a dash-dotted line, respectively. FIG. 9(a) and Table 1 present the results when the axial length is 23 mm, and FIG. 9(b) and Table 2 present the results when the axial length is 26 mm.

TABLE 1

| | |
|---|---|
| Spot diameter | Axial length: 23 mm |
| Cornea incident | Angle α [°] |

| diameter [mm] | 0 | 10 | 20 | 30 |
|---|---|---|---|---|
| 0.34 | 74.64 | 74.64 | 74.64 | 74.64 |
| 0.36 | 70.48 | 70.48 | 70.48 | 70.48 |
| 0.38 | 66.78 | 66.78 | 66.78 | 68.874 |
| 0.40 | 63.44 | 63.44 | 63.44 | 69.78 |
| 0.42 | 60.42 | 60.42 | 60.42 | 70.692 |
| 0.44 | 57.68 | 57.68 | 60.658 | 71.608 |
| 0.46 | 55.16 | 55.16 | 61.538 | 72.526 |

TABLE 2

| | |
|---|---|
| Spot diameter | Axial length: 26 mm |
| Cornea incident | Angle α [°] |

| diameter [mm] | 0 | 10 | 20 | 30 |
|---|---|---|---|---|
| 0.34 | 79.96 | 79.96 | 79.96 | 79.96 |
| 0.36 | 75.52 | 75.52 | 75.52 | 75.52 |
| 0.38 | 71.54 | 71.54 | 71.54 | 71.54 |
| 0.40 | 67.96 | 67.96 | 71.574 | 67.96 |
| 0.42 | 64.74 | 65.068 | 72.916 | 66.9 |
| 0.44 | 61.78 | 66.95 | 74.264 | 67.608 |
| 0.46 | 59.1 | 68.834 | 75.616 | 68.322 |

As presented in FIG. 9(a) and FIG. 9(b) and Table 1 and Table 2, in the case that the cornea incident diameter of the light beam 40 is 0.34 mm, the spot diameter of the light beam 40 is 79.96 μm when the axial length is 26 mm, and the spot diameter of the light beam 40 deviates from the range of 55 μm to 77 μm. When the cornea incident diameter of the light beam 40 is 0.46 mm, the variation in the spot diameter of the light beam 40 is the largest, the minimum spot diameter is 55.16 μm, the maximum spot diameter is 75.616 μm, and the variation is 1.37 dB.

From the results of FIG. 9(a) and FIG. 9(b) and Table 1 and Table 2, it is found that the cornea incident diameter of the light beam 40 is preferably adjusted to 0.36 mm to 0.46 mm (0.41 mm+0.05 mm) so that the spot diameter of the light beam 40 falls within a range of 55 μm to 77 μm. It is understood that, in order to reduce the variation in the spot diameter of the light beam 40, the cornea incident diameter of the light beam 40 is preferably 0.36 mm to 0.44 mm, more preferably 0.38 mm to 0.44 mm, and further preferably 0.38 mm to 0.42 mm.

As described above, according to the first embodiment, when the half angle θ of the larger one of the horizontal angle of view and the vertical angle of view of the area where the retina 62 is irradiated with a plurality of the light beams 40 at the convergence point 46 in the eye 60 is 10° or greater and 30° or less, the cornea incident diameters of the light beams 40 are adjusted to 0.36 mm or greater and 0.46 mm or less. Thus, even when the axial lengths (visual acuity) of the users are different, the amounts of variations in the spot diameters of the light beams 40 are kept small in the area where the retina 62 is irradiated with the light beams 40, and the spot diameters of the light beams 40 can be kept within a range of 55 μm or greater and 77 μm or less. Therefore, the user can obtain a sense of uniform resolution for the image projected on the retina 62.

In the first embodiment, the half angle θ of the larger one of the horizontal angle of view and the vertical angle of view of the area where the retina 62 is irradiated with the light beams 40 at the convergence point 46 in the eye 60 is 10° or greater and 30° or less. However, when the half angle θ of the angle of view is 10° or greater and the spot diameters of the light beams 40 are 55 μm or greater and 77 μm or less, the upper limit of the half angle θ of the angle of view may be larger than 30°.

According to the first embodiment, the numerical apertures when the light beams 40 enter the cornea 66 of the user are approximately zero regardless of the user. Thus, by adjusting the cornea incident diameters of the light beams 40 to 0.36 mm or greater and 0.46 mm or less, the spot diameters of the light beams 40 can be kept within a range of 55 μm or greater and 77 μm or less. The term “approximately zero” means-0.0005 or greater and +0.0005 or less.

According to the first embodiment, the light beams 40 are monochromatic light beams of red laser beams, green laser beams, or blue laser beams, or combined light beams obtained by combining at least two light beams of a red laser beam, a green laser beam, and a blue laser beam. In this case, the spot diameters of the light beams 40 can be kept within a range of 55 μm or greater and 77 μm or less by adjusting the diameters of the light beams 40 when entering the cornea 66 to 0.36 mm or greater and 0.46 mm or less.

In the first embodiment, in order to reduce variations in the spot diameters of the light beams 40, the cornea incident diameters of the light beams 40 are preferably 0.38 mm or greater and 0.44 mm or less. In this case, the cornea incident diameters of the light beams 40 are within a range of ±0.03 mm from a median of 0.41 mm in actual projection.

In the first embodiment, the image projection device 100 is attached to the spectacle-type frame 42. However, the frame is not limited to the spectacle-type frame, and may be a goggle-type frame, an eye patch-type frame, an ear-hung frame, a helmet-mounted frame, or the like as long as the frame can be mounted on the face of the user and the image projection device 100 can be installed in front of the eyes of the user.

### SECOND EMBODIMENT

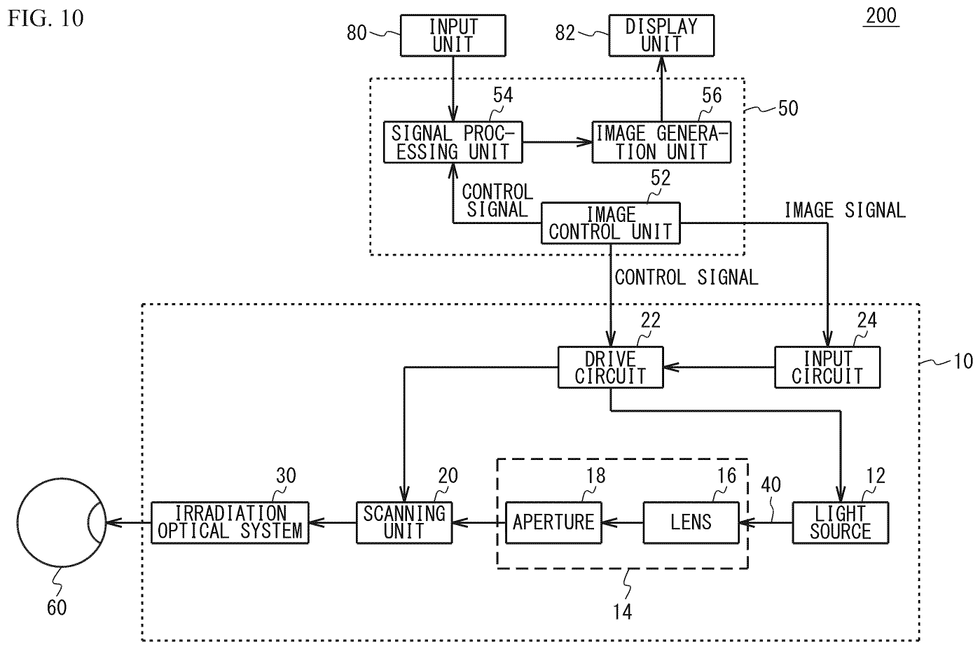

FIG. 10 is a block diagram of a vision test device 200 according to a second embodiment. As illustrated in FIG. 10, the vision test device 200 differs from FIG. 1 of the first embodiment in that the control unit 50 includes a signal processing unit 54 and an image generation unit 56 in addition to the image control unit 52. The image control unit 52 generates an image for testing to be projected onto the retina 62 of a subject. The signal processing unit 54 processes the response signal from an input unit 80 based on the control signal from the image control unit 52. The input unit 80 is a device for the subject to input a response signal during the vision test, and is, for example, a button, but may be another device. The image generation unit 56 generates a test result image based on the signal processed by the signal processing unit 54. A display unit 82 displays the test result image. The display unit 82 is, for example, a liquid crystal display.

The image control unit 52, the signal processing unit 54, and the image generation unit 56 may be implemented by a processor such as a CPU working in cooperation with a program. The image control unit 52, the signal processing unit 54, and the image generation unit 56 may be circuits designed for exclusive use. The image control unit 52, the signal processing unit 54, and the image generation unit 56 may be one circuit or different circuits.

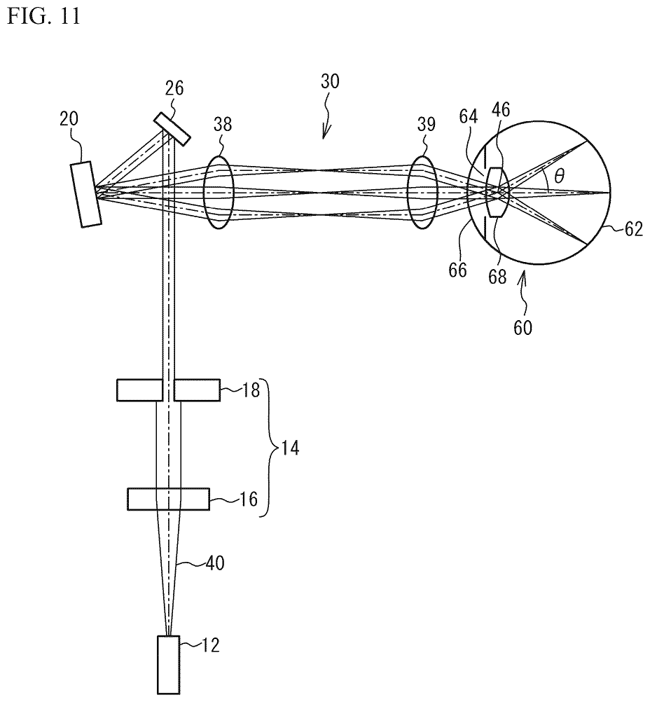

FIG. 11 illustrates an optical system of the vision test device 200 according to the second embodiment. As illustrated in FIG. 11, the vision test device 200 projects an image for testing onto the retina 62 using Maxwellian view. The light beam 40 emitted from the light source 12 is converted from diffusion light into substantially parallel light by the lens 16. The diameter of the light beam 40 transmitted through the lens 16 is adjusted by the aperture 18. The light beam 40 that has passed through the aperture 18 is reflected by a plane mirror 26 and enters the scanning unit 20.

A plurality of the light beams 40, which are scanned in the two-dimensional directions by the scanning unit 20 and are emitted from the scanning unit 20 in different directions at different times, enter the irradiation optical system 30. The irradiation optical system 30 includes a lens 38 and a lens 39. The light beams 40 enter the eye 60 of the subject through the lens 38 and the lens 39. The lens 38 is, for example, a condenser lens. The light beams 40 are made substantially parallel to each other by the lens 38, and each of the light beams 40 is converted from substantially parallel light into convergent light by the lens 38. Each of the light beams 40 is condensed before the lens 39, becomes diffusion light, and enters the lens 39. The lens 39 is, for example, a condenser lens. Each of the light beams 40 is converted from diffusion light into substantially parallel light by the lens 39, and enters the eye 60 of the subject. Thus, the numerical aperture of each of the light beams 40 when entering the cornea 66 is approximately zero. This does not change depending on the subject who uses the vision test device 200. The diameters of the light beams 40 when entering the cornea 66 are 0.36 mm to 0.46 mm (0.41 mm±0.05 mm), as in the first embodiment. The light beams 40 converge at the convergence point 46 in the eye 60 of the subject. The convergence point 46 is located, for example, in or near a crystalline lens 68. Each of the light beams 40 is converted from parallel light to convergent light by the crystalline lens 68 and is focused in the vicinity of the retina 62. The half angle θ of the larger one of the horizontal angle of view and the vertical angle of view of the area where the retina 62 is irradiated with the light beams 40 at the convergence point 46 in the eye 60 is 10° or greater and 30° or less as in the first embodiment. The area where the retina 62 is irradiated with the light beams 40 is a region where a test image for testing the visual function of the subject is projected.

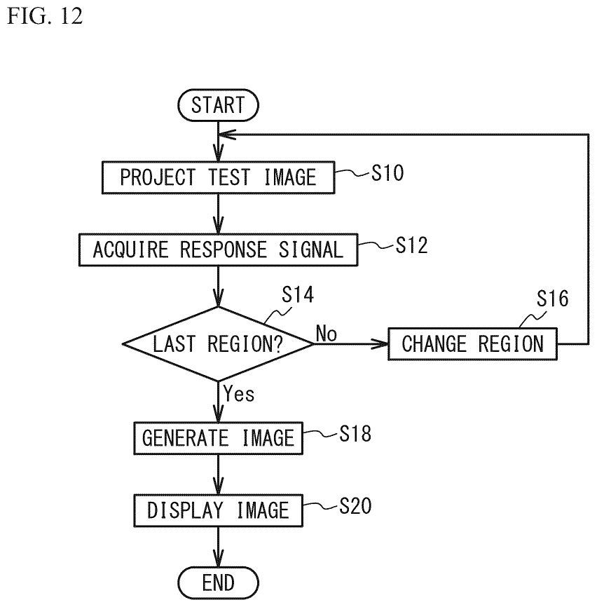

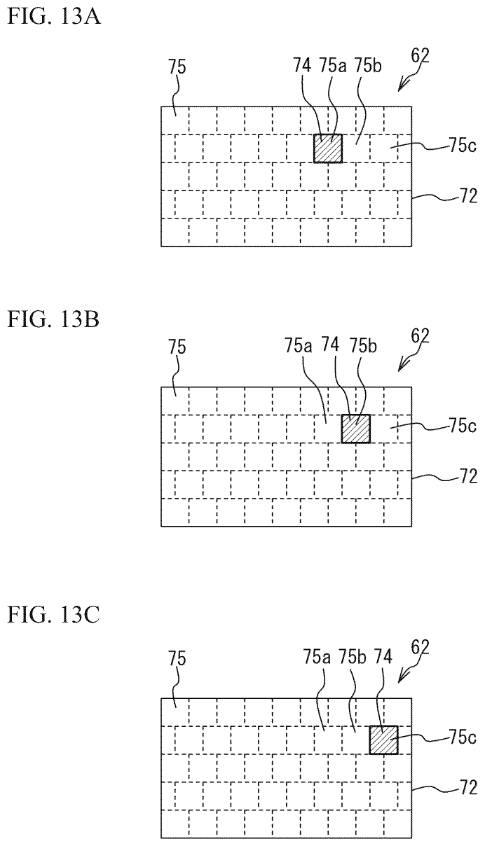

FIG. 12 is a flowchart illustrating an example of a testing method of the vision test device 200 according to the second embodiment. FIG. 13(a) to FIG. 13(c) are diagrams illustrating the image 72 for testing projected on the retina 62 in the flowchart of FIG. 12. As illustrated in FIG. 12, the image control unit 52 causes the light source 12 to emit the light beam 40 to project the image 72 for testing including a test target 74 onto the retina 62 (step S10). The light beam 40 is a monochromatic light beam of a red laser beam, a green laser beam, or a blue laser beam, or a combined light beam obtained by combining at least two of a red laser beam, a green laser beam, and a blue laser beam. In step S10, as illustrated in FIG. 13(a), the image 72 for testing including the test target 74 projected in a region 75a is projected on the retina 62. In the second embodiment, a region 75 is a region where the retina 62 is irradiated with one light beam 40. The region 75 may be a region irradiated with a plurality of the light beams 40. Although not illustrated, the image 72 for testing may include a fixation target for directing the line of sight of the subject.

Then, the signal processing unit 54 acquires a response signal from the input unit 80 (step S12). When the subject detects that the test target 74 is projected on the region 75a, the subject operates the input unit 80. Therefore, the signal processing unit 54 can acquire the response signal from the input unit 80 when the subject detects the test target 74, and cannot acquire the response signal from the input unit 80 when the subject cannot detect the test target 74.

After a predetermined time has elapsed from the projection of the image 72 for testing in step S10, the image control unit 52 determines whether the region is the last region (step S14). For example, when the testing of all the regions 75 to be tested in the retina 62 is completed, the determination becomes Yes. When the determination is No, the image control unit 52 changes the region 75 onto which the test target 74 is projected (step S16), and the process returns to step S10. The steps S10 to S16 are repeated until the determination becomes Yes in step S14. FIG. 13(b) illustrates a case where the region onto which the test target 74 is projected is changed to a region 75b, and FIG. 13(c) illustrates a case where the region onto which the test target 74 is projected is changed to a region 75c.

When the determination in step S14 is Yes, the image generation unit 56 generates an image of the test result of the visual function (for example, a visual field defect image) based on the response signal from the input unit 80 in each region 75 of the signal processing unit 54 (step S18). The display unit 82 displays the test result image (step S20).

According to the second embodiment, as in the first embodiment, when the half angle θ of the larger one of the horizontal angle of view and the vertical angle of view of the area where the retina 62 is irradiated with a plurality of the light beams 40 at the convergence point 46 in the eye 60 is 10° or greater and 30° or less, the cornea incident diameters of the light beams 40 are adjusted to 0.36 mm or greater and 0.46 mm or less. Thus, even when the axial lengths (visual acuity) of the subjects are different, the amounts of variations in the spot diameters of the light beams 40 can be kept small within the area where the retina 62 is irradiated with the light beams 40, and the spot diameters of the light beams 40 can be kept within a range of 55 μm or greater and 77 μm or less. Therefore, the visual function test can be performed on the subjects having different axial lengths over a wide area of the retina 62 under the same conditions, and the accuracy of the visual function test can be improved.

In the second embodiment, the half angle θ of the larger one of the horizontal angle of view and the vertical angle of view of the area where the retina 62 is irradiated with a plurality of the light beams 40 at the convergence point 46 in the eye 60 is 10° or greater and 30° or less. However, as in the first embodiment, when the half angle θ of the angle of view is 10° or greater and the spot diameters of the light beams 40 are 55 μm or greater and 77 μm or less, the upper limit of the half angle θ of the angle of view may be larger than 30°.

According to the second embodiment, the region 75 in FIG. 13(a) to FIG. 13(c) is a region where the retina 62 is irradiated with one light beam 40. Therefore, the subject operates the input unit 80 to respond to each of the light beams 40 sequentially applied to the retina 62. Since the spot diameters of the light beams 40 are within the predetermined range, the accuracy of the visual function test in which the subject responds to each of the light beams 40 is improved.

According to the second embodiment, the light beams 40 are monochromatic light beams of red laser beams, green laser beams, or blue laser beams, or combined light beams obtained by combining at least two of a red laser beam, a green laser beam, and a blue laser beam. In this case, as described in the first embodiment, the spot diameters of the light beams 40 can be kept within a range of 55 μm or greater and 77 μm or less by adjusting the diameters of the light beams 40 when entering the cornea 66 to 0.36 mm or greater and 0.46 mm or less. When the spot diameters of the light beams 40 are within such a range, the accuracy of the visual function test is improved.

In addition, in the second embodiment, as in the first embodiment, the numerical apertures when a plurality of the light beams 40 enter the cornea 66 of the subject are approximately zero regardless of the subject. Thus, by adjusting the cornea incident diameters of the light beams 40 to 0.36 mm or greater and 0.46 mm or less, the spot diameters of the light beams 40 can be kept within a range of 55 μm or greater and 77 μm or less.

In the second embodiment, as in the first embodiment, the cornea incident diameters of the light beams 40 are preferably 0.38 mm or greater and 0.44 mm or less (0.41 mm+0.03 mm) in order to reduce variations in the spot diameters of the light beams 40.

### THIRD EMBODIMENT

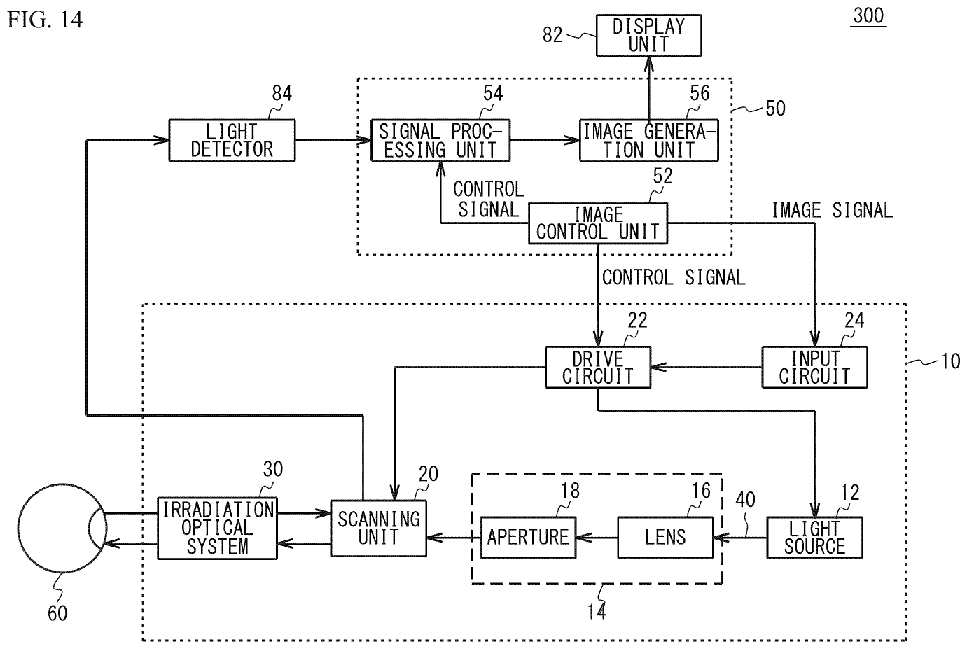

FIG. 14 is a block diagram of a fundus photography device 300 according to a third embodiment. As illustrated in FIG. 14, in the fundus photography device 300, the control unit 50 includes the signal processing unit 54 and the image generation unit 56 in addition to the image control unit 52, as in the second embodiment. The image control unit 52 generates an image for testing to be projected onto the retina 62 of the subject. The signal processing unit 54 processes the output signal of a light detector 84 based on the control signal from the image control unit 52. The light detector 84 detects the light beam 40 reflected by the retina 62. The light detector 84 includes an image sensor such as a CMOS image sensor or a CCD image sensor. The image generation unit 56 generates a fundus image based on a signal obtained by the signal processing unit 54 processing the output signal of the light detector 84. The display unit 82 displays the fundus image.

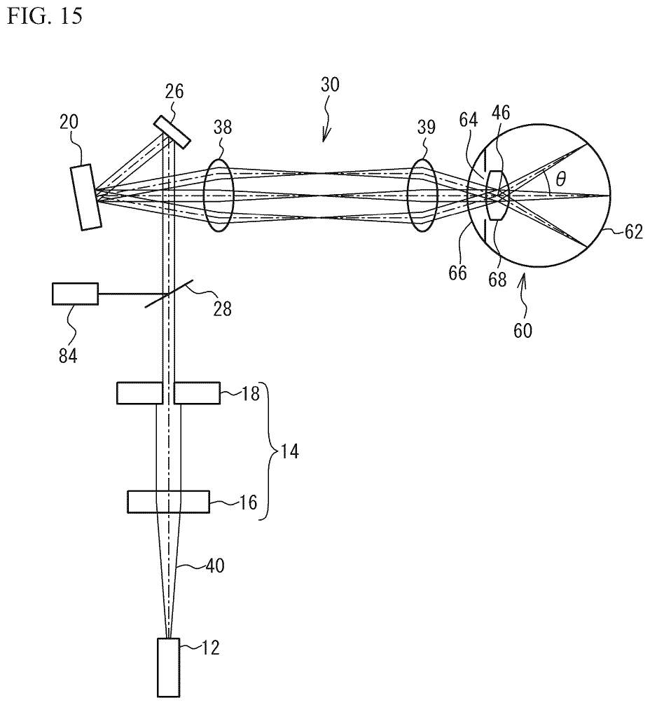

FIG. 15 illustrates an optical system of the fundus photography device 300 according to the third embodiment. As illustrated in FIG. 15, the fundus photography device 300 irradiates the retina 62 with the light beam 40 using Maxwellian view, as in the second embodiment. A half mirror 28 is provided on the optical path of the light beam 40 between the aperture 18 and the plane mirror 26. The light beam 40 reflected by the retina 62 enter the half mirror 28 via the lens 39, the lens 38, the scanning unit 20, and the plane mirror 26, is reflected by the half mirror 28, and enters the light detector 84. Other configurations are the same as those of the second embodiment illustrated in FIG. 11, and therefore, the description is omitted. In the third embodiment, the numerical aperture of each of a plurality of the light beams 40 when the light beams 40 enter the cornea 66 is approximately zero. This does not change depending on the subject who uses the fundus photography device 300. The diameters of the light beams 40 when entering the cornea 66 are 0.36 mm to 0.46 mm (0.41 mm+0.05 mm). The half angle θ of the larger one of the horizontal angle of view and the vertical angle of view of the area where the retina 62 is irradiated with the light beams 40 at the convergence point 46 in the eye 60 is 10° or greater and 30° or less as in the first embodiment.

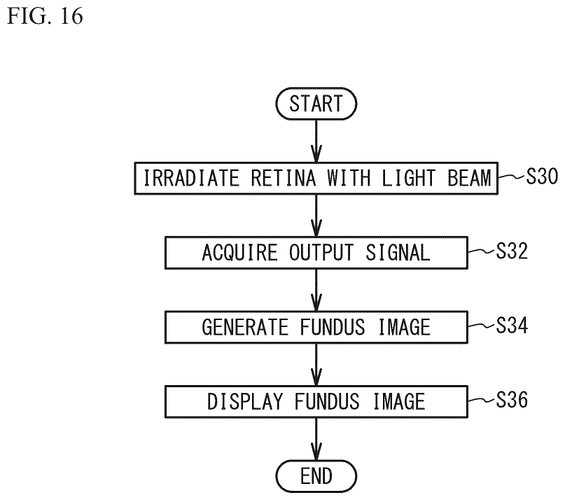

FIG. 16 is a flowchart illustrating an example of a testing method of the fundus photography device 300 according to the third embodiment. As illustrated in FIG. 16, the image control unit 52 causes the light source 12 to emit the light beam 40 for fundus photography, and causes the retina 62 to be irradiated with the light beam 40 (step S30). The light beam 40 for fundus photography is a monochromatic light beam of a red laser beam, a green laser beam, or a blue laser beam, or a combined light beam obtained by combining at least two of a red laser beam, a green laser beam, and a blue laser beam. The light beam 40 may be an invisible light beam such as an infrared laser beam. The light beam 40 is raster-scanned from the upper left to the lower right on the retina 62 and is applied onto the retina 62. The light beam 40 reflected by the retina 62 enters the light detector 84 as described in FIG. 15.

The signal processing unit 54 acquires the output signal from the light detector 84 (step S32). The image generation unit 56 generates a fundus image based on the signal obtained by the signal processing unit 54 processing the output signal from the light detector 84 (step S34). The display unit 82 displays the fundus image generated by the image generation unit 56 (step S36).

According to the third embodiment, as in the first embodiment, when the half angle θ of the larger one of the horizontal angle of view and the vertical angle of view of the area where the retina 62 is irradiated with a plurality of the light beams 40 at the convergence point 46 in the eye 60 is 10° or greater and 30° or less, the cornea incident diameters of the light beams 40 are adjusted to 0.36 mm or greater and 0.46 mm or less. Thus, even when the subjects have different axial lengths (visual acuity), the amounts of variations in the spot diameters of the light beams 40 can be kept small in the area where the retina 62 is irradiated with the light beams 40, and the spot diameters of the light beams 40 can be kept within a range of 55 μm or greater and 77 μm or less. Therefore, it is possible to acquire fundus images of subjects having different axial lengths, which are captured under the same conditions over a wide area of the retina 62.

In the third embodiment, the half angle θ of the larger one of the horizontal angle of view and the vertical angle of view of the area where the retina 62 is irradiated with the light beams 40 at the convergence point 46 in the eye 60 is 10° or greater and 30° or less. However, as in the first embodiment, when the half angle θ of the angle of view is 10° or greater and the spot diameters of the light beams 40 are 55 μm or greater and 77 μm or less, the upper limit of the half angle θ of the angle of view may be larger than 30°.

According to the third embodiment, the light beams 40 are monochromatic light beams of red laser beams, green laser beams, or blue laser beams, or combined light beams obtained by combining at least two light beams of a red laser beam, a green laser beam, and a blue laser beam. In this case, as described in the first embodiment, the spot diameters of the light beams 40 can be kept within a range of 55 μm or greater and 77 μm or less by adjusting the diameters of the light beams 40 when entering the cornea 66 to 0.36 mm or greater and 0.46 mm or less. When the spot diameters of the light beams 40 are within such a range, it is possible to improve the accuracy of the fundus image.

In addition, in the third embodiment, as in the first embodiment, the numerical apertures when the light beams 40 enter the cornea 66 of the subject are approximately zero regardless of the subject. Thus, by adjusting the cornea incident diameters of the light beams 40 to 0.36 mm or greater and 0.46 mm or less, the spot diameters of the light beams 40 can be kept within a range of 55 μm or greater and 77 μm or less.

In the third embodiment, as in the first embodiment, the cornea incident diameters of the light beams 40 are preferably 0.38 mm or greater and 0.44 mm or less (0.41 mm±0.03 mm) in order to reduce variations in the spot diameters of the light beams 40.

In the first to third embodiments, the half angle θ of the larger one of the horizontal angle of view and the vertical angle of view of the area where the retina 62 is irradiated with the light beams 40 at the convergence point 46 in the eye 60 is 10° or greater and 30° or less. However, as illustrated in FIG. 5(a), since the variations in the spot diameters of the light beams 40 tend to increase as the angle α increases, the cornea incident diameters of the light beams 40 are preferably adjusted to 0.36 mm or greater and 0.46 mm or less when the half angle θ of the larger one of the horizontal angle of view and the vertical angle of view is 15° or greater. When the half angle θ of the angle of view is 20° or greater, the cornea incident diameters of the light beams 40 are more preferably adjusted to 0.36 mm or greater and 0.46 mm or less. The cornea incident diameters of the light beams 40 are more preferably adjusted to 0.36 mm or greater and 0.46 mm or less when the half angle θ of the angle of view is 25° or greater.

In the first to third embodiments, the area where the retina 62 is irradiated with the light beams 40 has a rectangular shape in which the length in the horizontal direction is longer than the length in the vertical direction. However, the area may have a rectangular shape in which the length in the vertical direction is longer than the length in the horizontal direction, or may have a circular shape, an elliptical shape, or the like. When the area where the retina 62 is irradiated with the light beams 40 has a circular shape, the half angle θ of the larger one of the horizontal angle of view and the vertical angle of view is the half angle of the angle of view in the diameter, and when the area has an elliptical shape, the half angle θ is the half angle of the angle of view in the major axis.

Although embodiments of the present invention have been described so far, the present invention is not limited to those particular embodiments, and various changes and modifications may be made to them within the scope of the invention claimed herein.

1. An image projection device comprising:
a light source;
a scanning unit that scans a light beam emitted from the light source; and
an optical system that converges a plurality of light beams, which are emitted from the scanning unit at different times, at a convergence point in an eye of a user and then irradiates a retina of the user with the plurality of light beams to project an image,
wherein diameters of the plurality of light beams incident on a cornea of the user are adjusted to 0.36 mm or greater and 0.46 mm or less when a half angle of a larger one of a horizontal angle of view and a vertical angle of view of an area where the retina is irradiated with the plurality of light beams at the convergence point is 10° or greater and 30° or less.

2. The image projection device according to claim 1, wherein the plurality of light beams have diameters of 55 μm or greater and 77 μm or less on the retina.

3. The image projection device according to claim 1, wherein numerical apertures of the plurality of light beams when entering the cornea of the user are approximately zero regardless of the user.

4. The image projection device according to claim 1, wherein the plurality of light beams incident on the cornea of the user have diameters of 0.38 mm or greater and 0.44 mm or less.

5. The image projection device according to claim 1, wherein the plurality of light beams are monochromatic light beams of red light beams, green light beams, or blue light beams, or combined light beams obtained by combining at least two of a red light beam, a green light beam, and a blue light beam.

6. A vision test device comprising:
a light source;
a scanning unit that scans a light beam emitted from the light source;
an optical system that converges a plurality of light beams, which are emitted from the scanning unit at different times, at a convergence point in an eye of a subject and then irradiates the retina of the subject with the plurality of light beams; and
an input unit to which a response of the subject to the plurality of light beams applied to the retina is input,
wherein diameters of the plurality of light beams are adjusted to 0.36 mm or greater and 0.46 mm or less when a half angle of a larger one of a horizontal angle of view and a vertical angle of view of an area where the retina is irradiated with the plurality of light beams at the convergence point is 10° or greater and 30° or less.

7. The vision test device according to claim 6, wherein the plurality of light beams have diameters of 55 μm or greater and 77 μm or less on the retina.

8. The vision test device according to claim 6, wherein the subject responds to each of the plurality of light beams sequentially applied to the retina by operating the input unit.

9. A fundus photography device comprising:
a light source;
a scanning unit that scans a light beam emitted from the light source;
an optical system that converges a plurality of light beams, which are emitted from the scanning unit at different times, at a convergence point in an eye of a subject and then irradiates a retina of the subject with the plurality of light beams;
a detector that detects the plurality of light beams reflected by the retina; and
an acquisition unit that acquires a fundus image of the subject from the plurality of light beams detected by the detector,
wherein, when a half angle of a larger one of a horizontal angle of view and a vertical angle of view of an area where the retina is irradiated with the plurality of light beams at the convergence point is 10° or greater and 30° or less, diameters of the plurality of light beams incident on the cornea of the subject are adjusted to 0.36 mm or greater and 0.46 mm or less.

10. The fundus photography device according to claim 9, wherein the plurality of light beams have diameters of 55 μm or greater and 77 μm or less on the retina.
